(19) United States
(12) Patent Application Publication
(10) Pub. No.: US 2004/0091897 A1
(43) Pub. Date: May 13, 2004

(54) Disease associated polymorphism in the ctla-4 locus

(76) Inventors: John Todd, Cambridgeshire (GB); Stephen Gough, Warwickshire (GB); Hironori Ueda, Cambridgeshire (GB)

Correspondence Address: NIXON & VANDERHYE, PC, 1100 N GLEBE ROAD, 8TH FLOOR, ARLINGTON, VA, 22201-4714, US

(21) Appl. No.: 10/472,175

(22) PCT Filed: Mar. 18, 2002
(86) PCT No.: PCT/GB02/01261

(51) Int. Cl. C12Q 1/68

(52) U.S. Cl. 435/6

(57) ABSTRACT

The present invention relates to the identification of Single Nucleotide Polymorphisms (Sips) and haplotypes in the CTLA-4 locus and their association with a predisposition, susceptibility or resistance to autoimmune disease such as Graves Disease (GD) or Type 1 Diabetes Mellitus.(TIDM)

The present invention relates to the identification of Single Nucleotide Polymorphisms (SNPs) in the CTLA-4 locus and the association of these SNPs with a predisposition, susceptibility or resistance to autoimmune disease.

Autoimmune disease affects 4% of European populations and includes organ-specific disorders such as Grave's disease (GD), type 1 diabetes mellitus (T1DM: 0.4% of European populations), Hashimoto's thyroidism, Addison's disease, rheumatoid arthritis and multiple sclerosis.

CTLA-4 (cytotoxic T lymphocyte associated-4) is a candidate gene for T cell mediated autoimmune disease because it is a vital negative regulator of T cell activation. The CTLA-4 gene has been suggested as a candidate for conferring susceptibility to autoimmune diseases such as T1DM (IDDM12) and GD (Nistico L et al (1996) Hum Mol Genet 5: 1075-1080).

A gene or locus which confers susceptibility to a disease condition may contain one or more sites at which polymorphisms exist. The presence of these polymorphisms leads to different alleles of the gene or locus, one of which may be associated with an increased susceptibility to a disease condition.

An individual who is susceptible to a disease condition may have a predisposition to that condition which places that individual at a higher risk of incurring the condition during their lifetime than the population as a whole. Although at a higher risk of doing so, a susceptible individual may, in fact, never incur the disease condition.

Conversely, an allele may confer protection from disease. For a disease gene with two alleles, it can be difficult to distinguish between the gene having a susceptibility allele or a protective allele, where the other allele is neutrally or positively associated with disease.

The most common type of genetic polymorphism is a variation in the identity of a nucleotide at a single position in the genomic sequence (SNP). A gene associated with a disease may contain a number of SNPs within either its coding or non-coding region. Disease association may be caused by a particular SNP or by a particular haplotype consisting of a number of SNPs.

An SNP in coding sequence may alter the sequence of a polypeptide, giving rise to a defective or variant isoform which may be associated with a disease condition. An SNP in non-coding sequence may also lead to a disease condition, for example, by altering the activity of an enhancer element which directs polypeptide expression. Alternatively, an SNP may have no phenotypic effect.

Association analysis of particular SNPs and haplotypes using populations of affected and non-affected individuals may indicate that an SNP or haplotype is associated with a disease condition.

Three known SNPs within the CTLA-4 locus are shown in Table 2 (Deichmann et al (1996) Biochem. Biophys. Res. Commun. 225:817-818, Harper et al (1991) J. Immunol 147:1037-44, Marron et al (2000) Diabetes 49:492-499). Although some disease association has previously been noted for the 49G>A SNP (Table 2), this association is not sufficient to provide a meaningful diagnostic test for disease susceptibility in an individual because this SNP only seems to capture part of the association of the CTLA-4 locus with type 1 diabetes and Graves Disease and in some populations-is not associated at all with type 1 diabetes.

The present inventors have discovered a number of novel SNPs in the CTLA-4 locus and shown that both individual SNPs and haplotypes are strongly associated with disease conditions. This allows the development of methods associated with the diagnosis and therapy of CTLA-4 related autoimmune conditions.

A first aspect of the present invention provides a method for determining the susceptibility of an individual to a T-cell associated autoimmune disorder comprising:

determining the identity of a nucleotide present at one or more positions of single nucleotide polymorphism within the CTLA-4 locus of a genomic DNA sequence obtained from an individual, said one or more positions being selected from the group consisting of;

positions -34563, -23327, -14953, -12527, -11534, -10914, -9914, -8916, -2871, -2201, -1765, -1577, 6230, 7092, 7134, 7982, 8173, 8857, 10242, 10717, 12311, 16558, 19178, 21660, 22616, 24212 of the CTLA-4 locus, wherein the nucleotide 5′ to the A of the ATG of the CTLA-4 initiation codon is designated -1 and the sequence of the CTLA-4 locus has the database accession number AF225900.

These SNPs are shown in Table 1 and SEQ ID NOS: 1 to 26.

The susceptibility of the individual to a T-cell mediated autoimmune disorder such as Grave's disease or Type 1 diabetes mellitus is indicated by the identity of the nucleotide present at said one or more positions.

The nucleotide at the one or more positions of single nucleotide polymorphism may be an allele which is shown in Table 1 and SEQ ID NOS: 1 to 26.

In some embodiments, the identity of nucleotides at positions of single nucleotide polymorphism at -23327 and 6230 of the CTLA-4 locus may be determined, in particular the presence of a haplotype which comprises nucleotide G at these positions.

In addition to determining the identity of nucleotides at these two positions of single nucleotide polymorphism, the identity of nucleotides at other positions of single nucleotide polymorphism described herein and shown in table 1 may also be determined. In particular, the identity of the nucleotide at -34563 may be determined, example, the presence or absence of the nucleotide T at this position.

In other embodiments, methods may comprise determining the identity of the nucleotide at the position of single nucleotide polymorphism at 6230 of the CTLA-4 locus, for example, the presence or absence of the nucleotide G at this position. In addition to determining the identity of a nucleotide at the position of single nucleotide polymorphism at 6230 of the CTLA-4 locus, the identity of nucleotides at other positions of single nucleotide polymorphism described herein and shown in table 1 may also be determined.

In other embodiments, methods may comprise determining the identity of the nucleotide at the position of single nucleotide polymorphism at -23327 of the CTLA-4 locus, for example, the presence or absence of the nucleotide G at this position. In addition to determining the identity of nucleotides at the position of single nucleotide polymorphism at -23327 of the CTLA-4 locus, the identity of nucleotides at other positions of single nucleotide polymorphism described herein and shown in table 1 may also be determined.

Previous reports (Perkins et al J. Immunol. (1996) 156:4154-4159) indicate that 335bp of CTLA4 sequence are sufficient to control the inducibility of the gene. Only the -319C>T SNP is within this region. However, the regulatory elements which modulate the transcription of CTLA-4 have not yet been established.

Particular alleles of polymorphisms which are located in the CTLA-4 regulatory regions may alter expression from the gene or affect the processing or stability of the mRNA transcript. The presence of such alleles may be determined by measuring the amount and/or stability of the CTLA-4 mRNA.

Methods according to some aspects of the present invention may include obtaining a genomic sample. A test sample of genomic nucleic acid may be obtained, for example, by extracting nucleic acid from cells or biological tissues or fluids, urine, saliva, faeces, a buccal swab, biopsy or preferably blood, of an individual or for pre-natal testing from the amnion, placenta or foetus itself. Various methods are known for determining the presence or absence in a test sample of a particular nucleic acid sequence, for example an nucleic acid sequence which has a particular nucleotide at a position of single nucleotide polymorphism, as shown in Table 1 and SEQ ID NOS: 1 to 26. Furthermore, having sequenced nucleic acid of an individual or sample, the sequence information can be retained and subsequently searched without recourse to the original nucleic acid itself. Thus, for example a sequence alteration or mutation may be identified by scanning a database of sequence information using a computer or other electronic means.

Alternatively, tests may be carried out on preparations containing genomic DNA, cDNA and/or MRNA. Testing cDNA or mRNA has the advantage of the complexity of the nucleic acid being reduced by the absence of intron sequences, but the possible disadvantage of extra time and effort being required in making the preparations. RNA is more difficult to manipulate than DNA because of the wide-spread occurrence of RN'ases.

Methods according to some aspects of the present invention may comprise determining the binding of a oligonucleotide probe to the genomic sample. The probe may comprise a nucleotide sequence which binds specifically to a particular allele of the at least one polymorphism and does not bind specifically to other alleles of the at least one polymorphism.

The oligonucleotide probe may comprise a label and binding of the probe may be determined by detecting the presence of the label. A method may include hybridisation of one or more (e.g. two) oligonucleotide probes or primers to target nucleic acid. Where the nucleic acid is double-stranded DNA, hybridisation will generally be preceded by denaturation to produce singlestranded DNA. The hybridisation may be as part of a PCR procedure, or as part of a probing procedure not involving PCR. An example procedure would be a combination of PCR and low stringency hybridisation. A screening procedure, chosen from the many available to those skilled in the art, is used to identify successful hybridisation events and isolated hybridised nucleic acid.

Binding of a probe to target nucleic acid (e.g. DNA) may be measured using any of a variety of techniques at the disposal of those skilled in the art. For instance, probes may be radioactively, fluorescently or enzymatically labelled. Other methods not employing labelling of probe include examination of restriction fragment length polymorphisms, amplification using PCR, RN'ase cleavage and allele specific oligonucleotide probing. Probing may employ the standard Southern blotting technique. For instance DNA may be extracted from cells and digested with different restriction enzymes. Restriction fragments may then be separated by electrophoresis on an agarose gel, before denaturation and transfer to a nitrocellulose filter. Labelled probe may be hybridised to the DNA fragments on the filter and binding determined. DNA for probing may be prepared from RNA preparations from cells.

Those skilled in the art are well able to employ suitable conditions of the desired stringency for selective hybridisation, taking into account factors such as oligonucleotide length and base composition, temperature and so on.

Suitable selective hybridisation conditions for oligonucleotides of 17 to 30 bases include hybridization overnight at 42EC in 6× SSC and washing in 6× SSC at a series of increasing temperatures from 42° C. to 65° C.

Other suitable conditions and protocols are described in Molecular Cloning: a Laboratory Manual: 2nd edition, Sambrook et al., 1989, Cold Spring Harbor Laboratory Press and Current Protocols in Molecular Biology, Ausubel et al. eds., John Wiley & Sons, 1992.

An oligonucleotide for use in nucleic acid amplification may be about 30 or fewer nucleotides in length (e.g. 18, 21 or 24). Generally specific primers are upwards of 14 nucleotides in length, but need not be than 18-20. Those skilled in the art are well versed in the design. of primers for use processes such as PCR. Various techniques for synthesizing oligonucleotide primers are well known in the art, including phosphotriester and phosphodiester synthesis methods.

Nucleic acid may also be screened using a variant- or allele-specific probe. Such a probe may correspond in sequence to a region of the CTLA-4 gene, or its complement, which contains one or more of the single nucleotide polymorphisms described herein, which are shown to be associated with autoimmune disease susceptibility. Under suitably stringent conditions, specific hybridisation of such a probe to test nucleic acid is indicative of the presence of the sequence alteration in the test nucleic acid. For efficient screening purposes, more than one probe may be used on the same test sample.

Nucleic acid in a test sample, which may be a genomic sample or an amplified region thereof, may be sequenced to identify or determine the identity of a polymorphic allele. An allele may be identified by comparing the sequence obtained with the sequence shown in any of the figures herein. The allele of the SNP in the test nucleic acid can therefore be compared with the susceptibility alleles of the SNP as described herein to determine, whether the test nucleic acid contains one or more alleles which are associated with GD, T1DM or other autoimmune disease.

Since it will not generally be time- or labour-efficient to sequence all nucleic acid in a test sample or even the whole CTLA-4 gene, a specific amplification reaction such as PCR using one or more pairs of primers may be employed to amplify the region of interest in the nucleic acid, for instance the CTLA-4 gene or a particular region of the CTLA-4 locus in which polymorphisms associated with autoimmune disease susceptibility occur. The amplified nucleic acid may then be sequenced as above, and/or tested in any other way to determine the presence or absence of a particular feature. Nucleic acid for testing may be prepared from nucleic acid removed from cells or in a library using a variety of other techniques such as restriction enzyme digest and electrophoresis.

Sequencing of an amplified product may involve precipitation with isopropanol, resuspension and sequencing using a TaqFS+ Dye terminator sequencing kit. Extension products may be electrophoresed on an ABI 377 DNA sequencer and data analysed using Sequence Navigator software.

Nucleic acid in a test sample may be probed under conditions for selective hybridisation and/or subjected to a specific nucleic acid amplification reaction such as the polymerase chain reaction (PCR) (reviewed for instance in “PCR protocols; A Guide to Methods and Applications”, Eds. Innis et al, 1990, Academic Press, New York, Mullis et al, Cold Spring Harbor Symp. Quant. Biol., 51:263, (1987), Ehrlich (ed), PCR technology, Stockton Press, NY, 1989, and Ehrlich et al, Science, 252:1643-1650, (1991)). PCR comprises steps of denaturation of template nucleic acid (if double-stranded), annealing of primer to target, and polymerisation. The nucleic acid probed or used as template in the amplification reaction may be genomic DNA, cDNA or RNA.

Other specific nucleic acid amplification techniques include strand displacement activation, the QB replicase system, the repair chain reaction, the ligase chain reaction, rolling circle amplification and ligation activated transcription. For convenience, and because it is generally preferred, the term PCR is used herein in contexts where other nucleic acid amplification techniques may be applied by those skilled in the art. Unless the context requires otherwise, reference to PCR should be taken to cover use of any suitable nucleic amplification reaction available in the art.

Methods of the present invention may therefore comprise amplifying the portion of the CTLA-4 gene locus and region in said genomic sample containing the one or more positions of single nucleotide polymorphism.

Allele- or variant-specific oligonucleotides may be used in PCR to specifically amplify particular sequences if present in a test sample. Assessment of whether a PCR band contains a gene variant may be carried out in a number of ways familiar to those skilled in the art. The PCR product may for instance be treated in a way that enables one to display the polymorphism on a denaturing polyacrylamide DNA sequencing gel, with specific bands that are linked to the gene variants being selected.

In some embodiments, the region of genomic sample comprising a polymorphism may be amplified using a pair of oligonucleotide primers, of which the first member of the pair comprises a nucleotide sequence which hybridises to a complementary sequence which is proximal to and 5′ of the position of single nucleotide polymorphism, and the second member of the primer pair comprises a nucleotide sequence which hybridises to a complementary sequence which is proximal to and 3′ of the position of single nucleotide polymorphism.

In other embodiments, the first member of the pair of oligonucleotide primers may comprise a nucleotide sequence which hybridises to a complementary sequence which is proximal to and 5′ or 3′ of the polymorphism, and the second member of the pair may comprise a nucleotide sequence which hybridises under stringent conditions to a particular allele of the polymorphism and not to other alleles, such that amplification only occurs in the presence of the particular allele.

A further aspect of the present invention provides a pair of oligonucleotide amplification primers suitable for use in the methods described herein.

A suitable pair of amplification primers according to this aspect may have a first member comprising a nucleotide sequence which hybridises to a complementary sequence which is proximal to and 5′ of a single nucleotide polymorphism in the CTLA-4 locus at position -34563, -23327, -14953, -12527, -11534, -10914, -9914, -8916, -2871, -2201, -1765, -1577, 6230, 7092, 7134, 7982, 8173, 8857, 10242, 10717, 12311, 16558, 19178, 21660, 22616, or 24212, for example as shown in Table 1 and SEQ ID NOS 1 to 26, and a second member comprising a nucleotide sequence which hybridises to a complementary sequence which is proximal to and 3′ of the single nucleotide polymorphism.

The allele of the at least one polymorphism (i.e. the identity of the nucleotide at the position of single nucleotide polymorphism) may then be determined by determining the binding of an oligonucleotide probe to the amplified region of the genomic sample. A suitable oligonucleotide probe comprises a nucleotide sequence which binds specifically to a particular allele of the at least one polymorphism and does not bind specifically to other alleles of the at least one polymorphism.

Other suitable pairs of amplification primers may have a first member comprising a nucleotide sequence which hybridises to a complementary sequence which is proximal to and 5′ or 3′ of a single nucleotide polymorphism at position -34563, -23327, -14953, -12527, -11534, -10914, -9914, -8916, -2871, -2201, -1765, -1577, 6230, 7092, 7134, 7982, 8173, 8857, 10242, 10717, 12311, 16558, 19178, 21660, 22616 or 24212 of the CTLA-4 locus, for example as shown in Table 1 and SEQ ID NOS: 1 to 26, and a second member of the pair comprising a nucleotide sequence which hybridises under stringent conditions to a particular allele of the polymorphism and not to other alleles, such that amplification only occurs in the presence of the particular allele.

An alternative or supplement to looking for the presence of variant sequences in a test sample is to look for the presence of the normal sequence, e.g. using a suitably specific oligonucleotide probe or primer. Use of oligonucleotide probes and primers has been discussed in more detail above.

A further aspect of the present invention provides an oligonucleotide which hybridises specifically to a nucleic acid sequence which comprises an allele of a polymorphism selected from the group consisting of single nucleotide polymorphisms at positions -34563, -23327, -14953, -12527, -11534, -10914,-9914,-8916,-2871,-2201,-1765,-1577, 6230, 7092, 7134, 7982, 8173, 8857, 10242, 10717, 12311, 16558, 19178, 21660, 22616 or 24212 of the CTLA-4 locus, for example as shown in Table 1 and SEQ ID NOS: 1 to 26.

Such oligonucleotides may be used in a method of screening nucleic acid. Some preferred oligonucleotides have a sequence which is complementary to a sequence shown in SEQ ID NOS 1 to 26, or a sequence which differs from such a sequence by addition, substitution, insertion or deletion of one or more nucleotides, but preferably without abolition of ability to hybridise selectively to an allele of a polymorphism as described herein, that is wherein the degree of similarity of the oligonucleotide or polynucleotide with one of the sequences given is sufficiently high.

In some preferred embodiments, oligonucleotides according to the present invention are at least about 10 nucleotides in length, more preferably at least about 15 nucleotides in length, more preferably at least about 20 nucleotides in length. Oligonucleotides may be up to about 100 nucleotides in length, more preferably up to about 50 nucleotides in length, more preferably up to about 30 nucleotides in length. The boundary value ‘about X nucleotides’ as used above includes the boundary value ‘X nucleotides’.

Allele- or variant-specific oligonucleotide probes or primers according to embodiments of the present invention may be selected from those shown in SEQ ID NOS: 1 to 26 and Table 1. Approaches which rely on hybridisation between a probe and test nucleic acid and subsequent detection of a mismatch may be employed. Under appropriate conditions (temperature, pH etc.), an oligonucleotide probe will hybridise with a sequence which is not entirely complementary. The degree of base-pairing between the two molecules will be sufficient for them to anneal despite a mis-match. Various approaches are well known in the art for detecting the presence of a mismatch between two annealing nucleic acid molecules. For instance, RN'ase A cleaves at the site of a mis-match. Cleavage can be detected by electrophoresing test nucleic acid to which the relevant probe or probe has annealed and looking for smaller molecules (i.e. molecules with higher electrophoretic mobility) than the full length probe/test hybrid.

Thus, an oligonucleotide probe that has the sequence of a region of the normal CTLA-4 gene (either sense or anti-sense strand) in which the SNPs associated with Grave's Disease, T1DM or other autoimmune disease susceptibility as described herein are known to occur may be annealed to test nucleic acid and the presence or absence of a mis-match determined. Detection of the presence of a mis-match may indicate the presence in the test nucleic acid of a mutation associated with autoimmune or other disease susceptibility. On the other hand, an oligonucleotide probe that has the sequence of a region of the gene including a polymorphism associated with Grave's Disease, T1DM or other autoimmune disease susceptibility may be annealed to test nucleic acid and the presence or absence of a mis-match determined. The presence of a mis-match may indicate that the nucleic acid in the test sample has the normal sequence (the absence of a mis-match indicating that the test nucleic acid has the mutation). In either case, a battery of probes to different regions of the gene may be employed.

Nucleic acid according to the present invention, such as an oligonucleotide probe and/or pair of amplification primers, may be provided as part of a kit, e.g. in a suitable container such as a vial in which the contents are protected from the external environment. The kit may include instructions for use of the nucleic acid, e.g. in PCR and/or a method for determining the presence of nucleic acid of interest in a test sample. A kit wherein the nucleic acid is intended for use in PCR may include one or more other reagents required for the reaction, such as polymerase, nucleosides, buffer solution etc. The nucleic acid may be labelled. A kit for use in determining the presence or absence of nucleic acid of interest may include one or more articles and/or reagents for performance of the method, such as means for providing the test sample itself, e.g. a swab for removing cells from the buccal cavity or a syringe for removing a blood sample (such components generally being sterile).

Another aspect of the present invention provides a method for determining the presence or absence of an allele of a polymorphic nucleic acid sequence in a test sample comprising:

contacting a polymorphic nucleic acid sequence with a probe which specifically binds to the allele of the polymorphic nucleic acid sequence; and, determining binding of the nucleic acid sequence and the probe,

said method being characterised in that the polymorphic nucleic acid sequence comprises one or more positions of single nucleotide polymorphism selected from the group consisting of positions -34563, -23327, -14953, -12527, -11534, -10914, -9914, -8916, -2871, -2201, -1765, -1577, 6230, 7092, 7134, 7982, 8173, 8857, 10242, 10717, 12311, 16558, 19178, 21660, 22616 and 24212 of the CTLA-4 locus, wherein the nucleotide 5′ to the A of the ATG of the CTLA-4 initiation codon is designated -1 and the sequence of the CTLA-4 locus has the database accession number AF225900,

the identity of the nucleotide at the one or more positions of single nucleotide polymorphism determining the allele of the polymorphic nucleic acid sequence.

Another aspect of the present invention provides a method for determining the presence or absence in a test sample of an allele of a polymorphic nucleic acid sequence comprising one or more positions of single nucleotide polymorphism, the method comprising:

determining the identity of the nucleotide at one or more positions of single nucleotide polymorphism selected from the group consisting of positions -34563, -23327, -14953, -12527, -11534, -10914, -9914, -8916, -2871, -2201, -1765, -1577, 6230, 7092, 7134, 7982, 8173, 8857, 10242, 10717, 12311, 16558, 19178, 21660, 22616 and 24212 of the CTLA-4 locus wherein the nucleotide 5′ to the A of the ATG of the CTLA-4 initiation codon is designated -1 and the sequence of the CTLA-4 locus has the database accession number AF225900,

the presence of the allele of the polymorphic nucleic acid sequence being determined by the identity of the nucleotide at the one or more positions of single nucleotide polymorphism.

Optionally, such a method may comprise amplifying the polymorphic nucleic acid sequence using a pair of oligonucleotide primers. As noted physical detection may be employed using for example hybridisation of a suitable probe, or a transcription factor or other agent that binds nucleic acid in a sequence-specific fashion, or detection may be performed in silico or using suitable data analysis techniques, e.g. on a computer.

The identity of the nucleotides at positions of single nucleotide polymorphism at -23327 and 6230 of the CTLA-4 locus may be determined using such a method, in particular, the presence of the nucleotide G at these positions

Aspects of the present invention will now be illustrated with reference to the accompanying figures and experimental exemplification, by way of example and not limitation. Further aspects and embodiments will be apparent to those of ordinary skill in the art. All documents mentioned in this specification are hereby incorporated herein by reference.

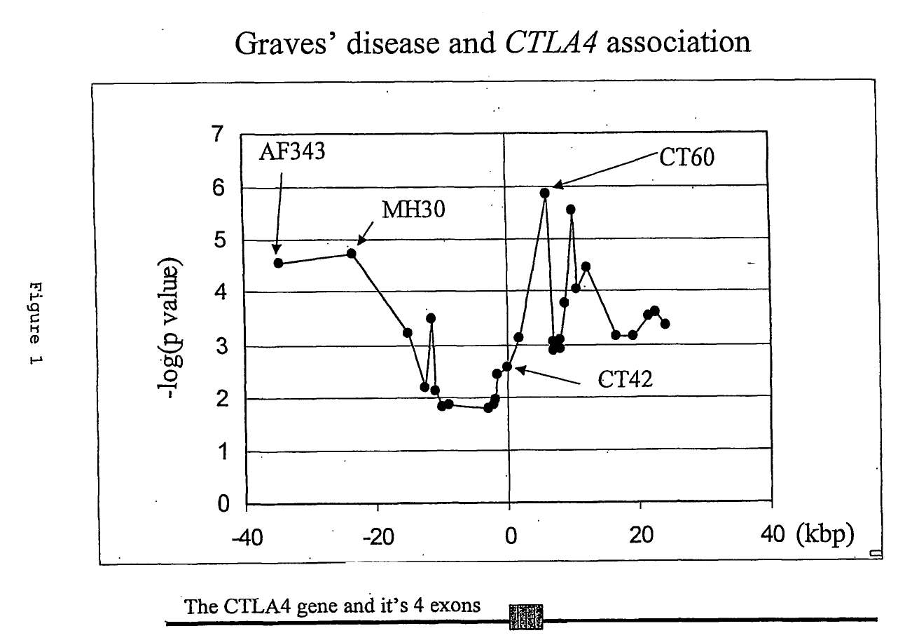

FIG. 1 shows association analysis for Graves disease. The location of SNPs in the CTLA-4 locus is shown on the x axis, using a single number to denote the position relative to the A of the ATG of the initiator codon. The y axis shows -log(pvalue). P value was calculated by X² test.

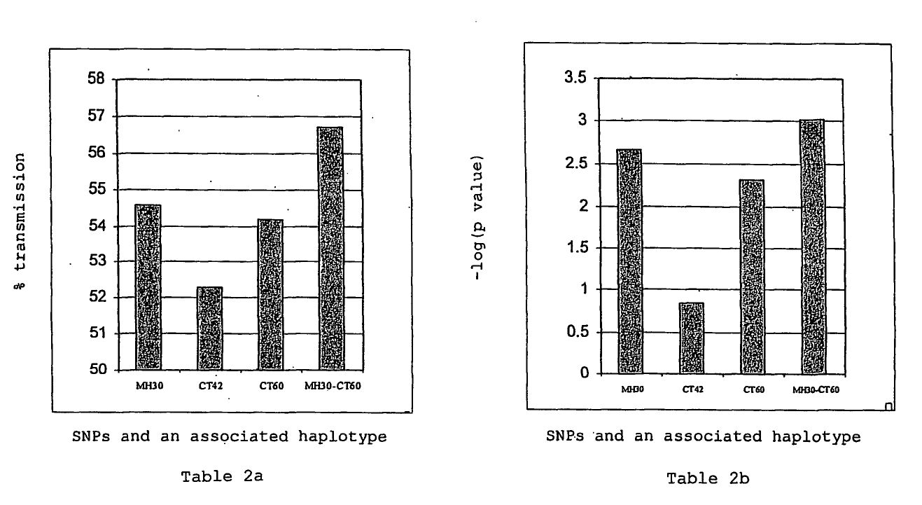

FIG. 2 shows association of the individual markers MH30 (-23327), CT42 (49) and CT60 (6230) and a haplotype of MH30 (23327) and CT60 (6230) in both UK and USA families with T1DM. In FIG. 2(a), the measure of association is the percentage transmission of an allele of a haplotype from parent to child (which is 50% in non-disease associated alleles or a haplotype). In FIG. 2(b), the -log(p value) is shown.

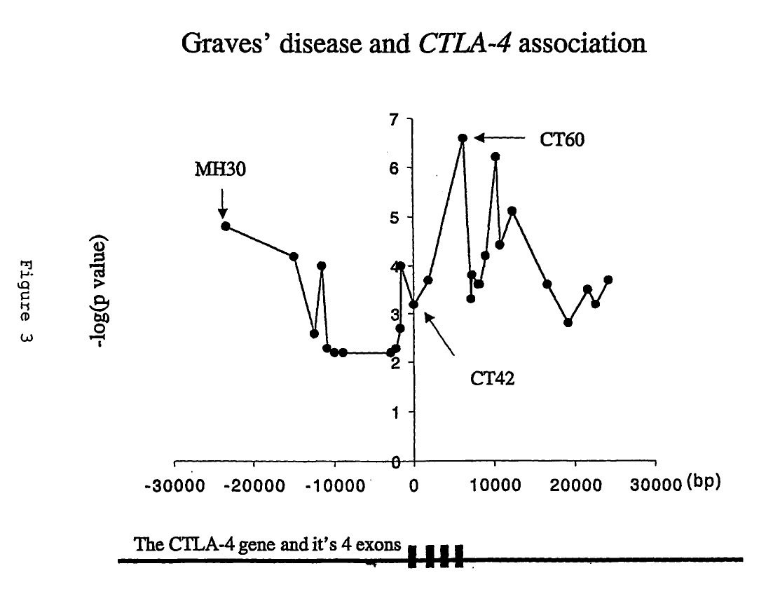

FIG. 3 corresponds to FIG. 1 and shows preliminary results of association analysis for Graves Disease.

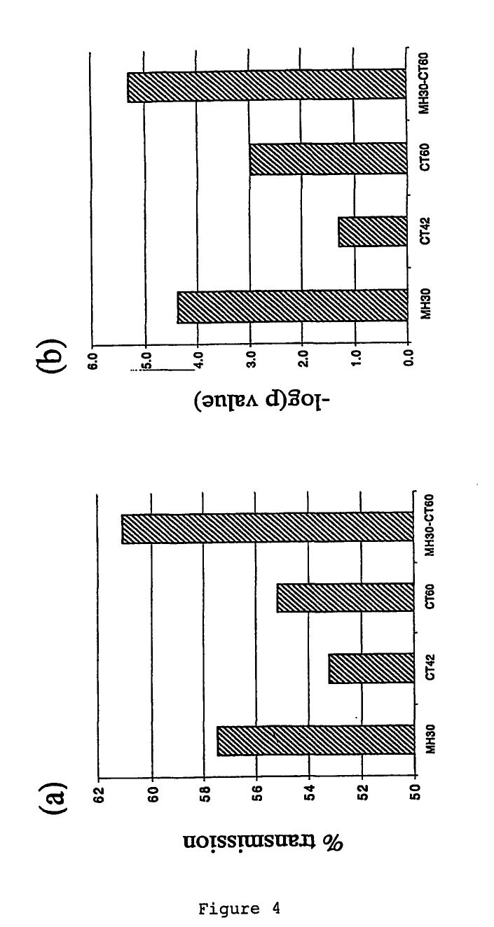

FIG. 4 corresponds to FIG. 2 and shows preliminary results of association analysis of the markers and haplotype for T1DM.

### EXPERIMENTAL

150 kb of the CTLA-4 region (primary sequence released into Genbank sequence database AF225900) have been sequenced by the present inventors to establish a dense map of SNPs in this region. The invention is not tied to the sequence of AF225900. Identification of SNP locations in similar sequences are contemplated. One skilled in the art can readily line up a similar sequence and located the same SNP locations (e.g., the SNP corresponding to position 6230 of AF225900 can be identified in a similar sequence by aligning the coding sequences).

A method for automated SNP harvesting using denaturing HPLC system was then applied. Eighty-seven 500 bp PCR fragments from a 48 kb region containing the CTLA-4 gene have been scanned by dHPLC in 32 individuals. Fragments that have different mobilities on dHPLC have been sequenced. This approach led us to identify more than 50 previously unknown SNPs in this region.

An SNP typing technology called the Invader chemistry (Third Wave Technologies) was then used. This uses a specific cleavage enzyme to recognise and cleave a reporter oligonucleotide bound to a polymorphic site (Mein CA et al (2000) Genome Research 10: 330-343).

The Invader system relies on the specificity of recognition and cleavage by a Flap endonuclease (FEN) of the three dimensional structure formed when two overlapping oligonucleotides, an Invader oligonucleotide and a signal oligonucleotide with a reporter arm hybridise to target DNA containing a polymorphic site (Lyamichev et al 1999 Nat. Biotechnol.17 292-296). Only in the presence of a perfect match between signal probe and template is the signal probe reporter arm or flap cleaved to drive a universal secondary cleavage reaction with a fluorescence energy transfer (FRET) label (Ryan et al (1999) Mol. Diagn. 4:135-144). Signal is detected at an end point with a conventional fluorescence microtitre plate reader.

References that may be consulted on the Invader technology include any one or more of the following, of which all are incorporated herein by reference: U.S. Pat. Nos. 5,846,717, 6,001,567, 5,985,557, 6,090,543, 5,994,069, 6,194,149, PCT applications WO 97/27214, WO 98/42873, WO 98/50403 and publications Lyamichev et al. Biochemistry 39, 9523-32 (2000), Hall et al. Proc Natl Acad Sci U S A 97, 8272-8277 (2000), Reynaldo J Mol Biol 297, 511-20 (2000), Griffin et al., Analytical Chemistry 72, 3298-3302 (2000).

References for cleavage enzymes that may be used in the assay include any one or more of the following, of which all are incorporated herein by reference: U.S. Pat. No. 5,541,311, 5,614,402, 5,795,763, 6,090,606, PCT applications WO 94/29482, WO 98/23774 and publications Lyamichev, et al. Nat Biotechnol 17, 292-6 (1999), Ma et al., J. Biol. Chem. 275 (32), 24693-24700 (2000), Lyamichev, et al., Proc Natl Acad Sci U S A 96, 6143-8 (1999) and Kaiser, et al., J. Biol. Chem. 274:21387 (1999).

Robust, high quality typing results were obtained on PCR products using this approach.

Forty five SNPs out of the fifty novel SNPs and previously reported three SNPs of the CTLA-4 region have been typed in 384 Graves' cases and 384-672 controls to ascertain the most disease associated region and begin to identify the aetiological polymorphisms. Several SNPs were found to show strong disease association.

Materials and Methods

DNA Samples

DNA was extracted from Epstein-Barr virus (EBV) transformed peripheral blood lymphocytes. Briefly, 100 ml of confluent EBV cells was pelleted at 1000 rpm for 5 mins. The supernatant was discarded and cells were resuspended in 4 ml of 5.25 guanidine hydrochloride (Sigma), 0.5 M ammonium acetate (Sigma), 125ng of proteinase K (sigma), and 1.3% sodium sarcosyl (Sigma). The solution was incubated overnight at 37° C. Two millilitres of chloroform was added and spun to 2500 rpm, the upper layer was removed and added to 10 ml of 100% ethanol, and the precipitated DNA was pelleted at 3000 rpm in an Allegra 6R micro-centrifuge (Beckman, UK). Pellets were washed with 70% ethanol and resuspended in 300 μl of Tris-EDTA (TE) (pH7.5). DNA was quantitated with Pico Green (Molecular Probes, Eugene OR) and diluted to 4 ng/μl in TE (pH 7.5) before use.

PCR

PCR conditions were optimised by varying MgCl₂ concentrations between 1 and 5 mM and annealing temperature between 50° C. and 65° C. PCRs were performed in 384 well polypropylene microtitre plates (Abgene, Epsom, Surrey, UK) in 6 μl final reaction volume. 3 μl of 4 ng/μl stock of genomic DNA was dispensed into each well with a Beckman Multimek™ 96 robot (Beckman, High Wycombe, UK) dedicated to pre-PCR work. 3μl of PCR reaction mix containing 0.4 mM dNTP, 2-10 mM MgCl₂, 3.8 ng/μl forward and reverse primer, and 0.25 units of TaqGold (Perkin Elmer Applied Biosystems, Foster City, Calif.) was added and the plate was covered with adhesive sealing sheet (Abgene, Epsom, Surrey, UK).

Reactions were incubated at 95° C. for 15 minutes and then cycled for 35 cycles of 95° C. for 30 sec, 50-65° C. for 30 and 72° C. for 30 sec and finally incubated for 15 mins at 72° C. on MJ PCT225 thermocyclers (MJ Research, Watertown, Mass.) using heated lids to prevent evaporation. All pipetting steps for PCR preparation were performed with a Beckman Multimek™ 96 robot (Beckman, High Wycombe, UK) dedicated to pre-PCR work.

Invader™ Assay

Probe sets for each locus were designed and synthesised by Third Wave Technologies, Inc (Madison, Wis.) (Lyamichev et al. 1999; Ryan et al. 1999) based on the sequence of the locus to be tested.

Assays were prepared for each allele separately. In the 384well format, PCR products were diluted 1 in 4 in distilled water. 3μl aliquots were dispensed into two pre-prepared 384-well format Invader® Assay FRET detection plates (Third Wave Technologies, Madison, Wis.). 6 μl of probe mix containing 1.3 μM allele specific probe (probe 1 or probe 2), 0.13 μM Invader™ probe and 7.5 mM MgCl₂ were added. All pipetting steps for this preparation were performed with a Multimek™ 96 robot (Beckman, High Wycombe, UK). Plates were covered with Adhesive Sealing Sheet (Abgene, Epsom, Surrey, UK). Reactions were incubated at 95° C. for 5 min, 65° C. for 10-80 min on MJ PTC225 thermocyclers (MJ Research).

Fluorescence was measured directly at the end of incubation using a Cytofluor 4400 fluorescence microtiter plate reader (Perkin Elmer Applied Biosystems, Foster City, Calif.), excitation 485/20, emission 530/25, and gain 50. Results were analysed using Excel software (Microsoft, Redmond, Wash.). Individual genotypes were scored by taking a ratio of signal strength from allele 1 and allele 2. The criteria for scoring genotypes varied between loci (see Results, but for most loci, an individual was typed as a heterozygote of the ratio of signal between the two alleles was between 0.5 and 2. ratios outside of this range were typed as homozygotes. An assay was classed as a failure if signals from both assays were below a threshold level dependent on locus or format.

Invader™ Biplex Assay

Invader™ Biplex assays (Third Wave Technologies, Inc. Madison, Wis.) were performed as described for the Invader uniplex assays above except that both allele specific probes (Probe 1 or Probe 2) were labeled with different fluorescent labels (FAM or RED). In the uniplex invader assay, both Probe 1 and Probe 2 were labelled by FAM. This enables both alleles to be detected in the same reaction so a single 384-well format Invader™ Assay FRET detection plate was used to type both alleles.

Tagman™ Assay

SNPs were also typed using the Taqman™ system (Perkin Elmer Applied Biosystem, Foster City, Calif.) and following standard protocols, as set out in the manufacturer's instructions and further described in Ranade K et al. (2001) Genome Res.11:1262-1268.

Restriction Fragment Length Polymorphism

Conventional Restriction Fragment Length Polymorphism (RFLP) analysis was performed as described in Sambrook J. and Russell D. Molecular Cloning: A Laboratory Manual (3rd Edition New York: Cold Spring Harbor Laboratory Press 2001 ISBN 0-87969-577-3) to type the following SNPs: CT50, CT53, CT42, CT55, and CT60 in Graves' cases and 40% of controls and CT42 in UK and USA TID families.

Single Nucleotide Results

Fifty one novel SNPs were identified in the CTLA-4 locus. Forty five of these SNPs were typed in the CTLA-4 locus, along with three previously reported SNPS, using the approach outlined above. In order to investigate the role of these SNPs in autoimmune disorders, the association of these SNPs with Graves disease was examined, along with the association of the three known SNPs in this locus at -319, 49 and 1822.

Of these SNPs, 28 showed (p<0.01) association with Graves Disease, including the two known SNPs at 49 and 1822. The known SNP at −319 does not show association. 26 novel SNPs have therefore been identified which show association with Graves disease (see Table 1).

The A of the ATG of the initiator Met codon of CTLA-4 is denoted nucleotide+1. The nucleotide 5′ to+1 is numbered -1 (Antonarakis SE et al. Hum Mutat. 1998;11:1-3). The location of an SNP in the CTLA-4 locus can therefore be precisely established using a single number denoting the position of the SNP relative to the A of the ATG of the initiator Met codon.

SNPs may also be named according to their position on contig AF225900.

Details of each of the SNPs analysed, along with the results of the disease association analysis, are provided below.

1) -34563T>C (AF343)

Position (bp) in contig AF225900: 34714

Sequence Region (SNP highlighted): atagcat gggagtattt tactgtgcta aaa[t/c]acattt agcatgggct gttatatctt atgactttga (SEQ ID NO: 1)

Allele frequencies;

| | Allele T | Allele C |
|---|---|---|
| Graves disease | 81.5 (613/752) | 18.5 (139/752) |
| Control | 73.4 (937/1276) | 26.6 (339/1276) |

Odds ratio = 1.6, χ² = 17.2, p = 2.7 × 10⁻⁵

The genotype frequencies;

| | TT | TC | CC |
|---|---|---|---|
| Graves' disease | 65.7% (247/376) | 31.6% (119/376) | 2.7% (10/376) |
| Control | 53.8% (343/638) | 39.3% (251/638) | 6.9% (44/638) |

genotype risk ratio = 1.65, χ² = 17.6, p = 1.5 × 10⁻⁴

2) -23327G>C (MH30)

Position (bp) in contig AF225900: 45950

Sequence Region (SNP in bold); aatgctcagttttatgacccaaaatcaatgaataaaaacagaataaaacaat[g/c]agaa aattttcacctttatttaattagcaga (SEQ ID NO:2)

The allele frequencies;

| | Allele G | Allele C |
|---|---|---|
| Graves' disease | 64.1% (477/744) | 35.9% (267/744) |
| Control | 54.4% (718/1320) | 45.6% (602/1320) |

odds ratio = 1.50, χ² = 18.4, p = 1.8 × 10⁻⁵

The genotype frequencies;

| | GG | GC | CC |
|---|---|---|---|
| Graves' disease | 41.9% (156/372) | 44.4% (165/372) | 13.7% (51/372) |
| Control | 29.7% (196/660) | 49.4% (326/660) | 20.9% (138/660) |

genotype risk ratio = 1.71, χ² = 18.4, p = 9.9 × 10⁻⁵

3) -14953G>T (MH26)

Position (bp) in contig AF225900: 54324

Sequence Region (SNP highlighted); tttctttcttttttggccccactgactctgtctcaagat[g/t]gtaattagtaactgaca atgattacgctatagttccatcatgaaaacat(SEQ ID NO:3)

The allele frequencies;

| | Allele T | Allele G |
|---|---|---|
| Graves' disease | 65.7% (503/766) | 34.3% (263/766) |
| Control | 56.7% (702/1238) | 43.3% (536/1238) |

odds ratio = 1.46, χ² = 15.9, p = 6.9 × 10⁻⁵

The genotype frequencies;

| | TT | TG | GG |
|---|---|---|---|
| Graves' disease | 42.3% (162/383) | 46.7% (179/383) | 11.0% (42/383) |
| Control | 33.0% (204/619) | 47.5% (294/619) | 19.5% (121/619) |

genotype risk ratio = 1.49, χ² = 16.4, p = 2.8 × 10⁻⁴

4) -12527G>A (MH20)

Position (bp) in contig AF225900: 56750

Sequence Region (SNP highlighted); actgtaaactgaaggtagtctgcctgacatgctt[g/a]gtgtatcttgtatgatttctgt aaagttagaaactgaggacatgcactca (SEQ ID NO:4)

The allele frequencies;

| | Allele G | Allele A |
|---|---|---|
| Graves' disease | 56.5% (428/758) | 43.5% (330/758) |
| Control | 63.3% (835/1320) | 36.7% (485/1320) |

odds ratio = 1.33, χ² = 9.3, p = 2.3 × 10⁻³

The genotype frequencies;

| | GG | GA | AA |
|---|---|---|---|
| Graves' disease | 29.6% (112/379) | 53.8% (204/379) | 16.6% (63/379) |
| Control | 38.5% (254/660) | 49.5% (327/660) | 12.0% (79/660) |

genotype risk ratio = 1.47, χ² = 10.1, p = 6.3 × 10⁻³

5) -11534T>C (MH18)

Position (bp) in contig AF225900: 57743

Sequence Region (SNP highlighted); gaagggaaaatacacttttaattaaaaattgttgagagttgaaagtaagaga[t/c]cctt cctaatagtgccttcttatctctcaggtg(SEQ ID NO:5)

The allele frequencies;

| | Allele T | Allele C |
|---|---|---|
| Graves' disease | 64.0% (488/762) | 36.0% (274/762) |
| Control | 55.3% (712/1288) | 44.7% (576/1288) |

odds ratio = 1.44, χ² = 15.1, p = 1.0 × 10⁻⁴

The genotype frequencies;

| | TT | TC | CC |
|---|---|---|---|
| Graves' disease | 40.2% (153/381) | 47.8% (182/381) | 12.1% (46/381) |
| Control | 30.4% (196/644) | 49.7% (320/644) | 19.9% (128/644) |

genotype risk ratio = 1.53, χ² = 15.4, p = 4.5 × 10⁻⁴

6) -10914T>C (MH17)

Position (bp) in contig AF225900: 58363

Sequence Region (SNP highlighted); aatcaatcaattttatttgctaaatttagtac [t/c]agagtgacattatctgtacattct ttg (SEQ ID NO:6)

The allele frequencies;

| | Allele T | Allele C |
|---|---|---|
| Graves' disease | 44.2% (333/754) | 55.8% (421/754) |
| Control | 37.8% (492/1300) | 62.2% (808/1300) |

odds ratio = 1.30, χ² = 7.9, p = 4.9 × 10⁻³

The genotype frequencies;

| | TT | TC | CC |
|---|---|---|---|
| Graves' disease | 17.8% (67/377) | 52.8% (199/377) | 29.4% (111/377) |
| Control | 13.2% (86/650) | 49.2% (320/650) | 37.5% (244/650) |

genotype risk ratio = 1.42, χ² = 8.4, p = 1.5 × 10⁻²

7) -9914G>T (MH15)

Position (bp) in contig AF225900: 59363

Sequence Region (SNP highlighted); aatgtcattgccatgacatggtcctattaggtgcatacagaaactgagctctatgc[g/t]tgtgccagacaaaaaccaaagagctt(SEQ ID NO:7)

The allele frequencies;

| | Allele T | Allele G |
|---|---|---|
| Graves' disease | 57.2% (435/760) | 42.8% (325/760) |
| Control | 63.3% (836/1320) | 36.7% (484/1320) |

odds ratio = 1.29, χ² = 7.5, p = 6.0 × 10⁻³

The genotype frequencies;

| | TT | TG | GG |
|---|---|---|---|
| Graves' disease | 30.8% (117/380) | 52.9% (201/380) | 16.3% (62/380) |
| Control | 39.5% (261/660) | 47.6% (314/660) | 12.9% (85/660) |

genotype risk ratio = 1.32, χ² = 8.5, p = 1.4 × 10⁻²

8) -8916G>A (MH13-1)

Position (bp) in contig AF225900: 60361

Sequence Region (SNP highlighted); tagcatgcaccttcattcctttttatggct [g/a]aataatattccgtggtgta gatagagtacattttgcttatccattcatc (SEQ ID NO:8)

The allele frequencies;

| | Allele G | Allele A |
|---|---|---|
| Graves' disease | 44.8% (337/752) | 55.2% (415/752) |
| Control | 38.6% (482/1250) | 61.4% (768/1250) |

odds ratio = 1.30, χ² = 8.0, p = 4.8 × 10⁻³

The genotype frequencies;

| | GG | GA | AA |
|---|---|---|---|
| Graves' disease | 17.3% (65/376) | 55.1% (207/376) | 27.7% (104/376) |
| Control | 13.9% (87/625) | 49.3% (308/625) | 36.8% (230/625) |

genotype risk ratio = 1.29, χ² = 9.2, p = 0.01

9) -2871G>A (MH2)

Position (bp) in.contig AF225900: 66406

Sequence Region (SNP highlighted); caccttaactttcaatgccttgatttccttctttataaaatgggaaaaatg[g/a]taact cttgtcttgtagggttgttatggacttgaaa (SEQ ID NO:9)

The allele frequencies;

| | Allele G | Allele A |
|---|---|---|
| Graves' disease | 44.3% (329/742) | 55.7% (413/742) |
| Control | 38.2% (493/1292) | 61.8% (799/1292) |

odds ratio = 1.29, χ² = 7.5, p = 6.2 × 10⁻³

The genotype frequencies;

| | GG | GA | AA |
|---|---|---|---|
| Graves' disease | 16.4% (61/371) | 55.8% (207/371) | 27.8% (103/371) |
| Control | 13.8% (89/646) | 48.8% (315/646) | 37.5% (242/646) |

genotype risk ratio = 1.23, χ² = 9.9, p = 6.9 × 10⁻³

10) -2201C>T (MH1)

Position (bp) in contig AF225900: 67076

Sequence Region (SNP highlighted); acagagttgagtagtggcaacagagaccc [c/t]accgtttgcaaatcataacatatttac tattttgcccccttcagaaagctttcca(SEQ ID NO:10)

The allele frequencies;

| | Allele T | Allele C |
|---|---|---|
| Graves' disease | 55.2% (402/728) | 44.8% (326/728) |
| Control | 61.6% (785/1274) | 38.4% (489/1274) |

odds ratio = 1.30, χ² = 7.9, p = 5.1 × 10⁻³

The genotype frequencies;

| | TT | TC | CC |
|---|---|---|---|
| Graves' disease | 27.7% (101/364) | 54.9% (200/364) | 17.3% (63/364) |
| Control | 37.2% (237/637) | 48.8% (311/637) | 14.0% (89/637) |

genotype risk ratio = 1.29, χ² = 9.5, p = 8.5 × 10⁻³

11) -1765T>C (CT50)

Position (bp) in contig AF225900: 67512

Sequence Region (SNP highlighted); ttccacaggctgaaccactggcttctgctcctctacataatacttcaa[t/c]tccagcat tgatctcactctatcatgatcatgggttta (SEQ ID NO:11).

The allele frequencies;

| | Allele T | Allele C |
|---|---|---|
| Graves' disease | 44.8% (343/766) | 55.2% (423/766) |
| Control | 38.1% (580/1522) | 61.9% (942/1522) |

odds ratio = 1.32, χ² = 9.4, p = 2.1 × 10⁻³

The genotype frequencies;

| | TT | TC | CC |
|---|---|---|---|
| Graves' disease | 17.8% (68/383) | 54.0% (207/383) | 28.2% (108/383) |
| Control | 13.5% (103/761) | 49.1% (374/761) | 37.3% (284/761) |

genotype risk ratio = 1.38, χ² = 10.4, p = 5.4 × 10⁻³

12) -1577G>A (CT53)

Position (bp) in contig AF225900: 67700

Sequence Region (SNP highlighted); gcccattaggttgttattgcttgttggcgcttgagctggggcttgaag[g/a]tttctata atgtgtagcagtgtatagaaaa (SEQ ID NO:12)

The allele frequencies;

| | Allele G | Allele A |
|---|---|---|
| Graves' disease | 64.7% (493/762) | 35.3% (269/762) |
| Control | 55.9% (671/1200) | 44.1% (529/1200) |

odds ratio = 1.44, χ² = 14.9, p = 1.1 × 10⁻⁴

The genotype frequencies;

| | GG | GA | AA |
|---|---|---|---|
| Graves' disease | 40.9% (156/381) | 47.5% (181/381) | 11.5% (44/381) |
| Control | 30.7% (184/600) | 50.5% (303/600) | 18.8% (113/600) |

genotype risk ratio = 1.57, χ² = 15.3, p = 4.9 × 10⁻⁴

13) 49G>A (CT42)

(Harper et al (1991) J.Immunol 147:1037-44.)

Position (bp) in contig AF225900: 69325

Sequence Region (SNP highlighted); tttcagcggcacaaggctcagctgaacctggct[g/a]ccaggacctggccctgcactctc ctgttttttcttctcttcatccc

The allele frequencies;

| | Allele G | Allele A |
|---|---|---|
| Graves' disease | 42.4% (326/768) | 57.6% (442/768) |
| Control | 35.1% (534/1522) | 64.9% (988/1522) |

odds ratio = 1.36, χ² = 1.1, p = 5.9 × 10⁻⁴

The genotype frequencies;

| | GG | GA | AA |
|---|---|---|---|
| Graves' disease | 16.9% (65/384) | 51.0% (196/384) | 32.0% (123/384) |
| Control | 11.8% (90/761) | 46.5% (354/761) | 41.7% (317/761) |

genotype risk ratio = 1.52, χ² = 12.1, p = 2.3 × 10⁻³

14) 1822T>C (CT55)

(Marron et al (2000) Diabetes 49:492-499)

Position (bp) in contig AF225900: 71098

Sequence Region (SNP highlighted); aggtaatttggcatgcagccactatttttgagttgatgcaag[t/c]ctctctgtatggag agctggtctcctttatcctgtgggaaaa

The allele frequencies;

| | Allele T | Allele C |
|---|---|---|
| Graves' disease | 42.3% (325/768) | 57.7% (443/768) |
| Control | 34.3% (511/1490) | 65.7% (979/1490) |

odds ratio = 1.41, χ² = 14.0, p = 1.8 × 10⁻⁴

The genotype frequencies;

| | TT | TC | CC |
|---|---|---|---|
| Graves' disease | 16.7% (64/384) | 51.3% (197/384) | 32.0% (123/384) |
| Control | 11.4% (85/745) | 45.8% (341/745) | 42.8% (319/745) |

genotype risk ratio = 1.55, χ² = 14.5, p = 7.2 × 10⁻⁴

15) 6230G>A (CT60)

Position (bp) in contig AF225900: 75506

Sequence Region (SNP highlighted); caagtcattcttggaaggtatccatcctctttccttttgatttcttcaccactatttggga tataac[g/a]tgggttaacacagacata (SEQ ID NO:13)

The allele frequencies;

| | Allele G | Allele A |
|---|---|---|
| Graves' disease | 62.9% (477/758) | 37.1% (281/758) |
| Control | 51.5% (736/1430) | 48.5% (694/1430) |

odds ratio = 1.60, χ² = 26.3, p = 2.9 × 10⁻⁷

The genotype frequencies;

| | GG | GA | AA |
|---|---|---|---|
| Graves' disease | 40.1% (152/379) | 45.6% (173/379) | 14.2% (54/379) |
| Control | 26.9% (192/715) | 49.2% (352/715) | 23.9% (171/715) |

genotype risk ratio = 1.82, χ² = 25.6, p = 2.6 × 10⁻⁶

16) 7092G>A (Jo37-3)

Position (bp) in contig AF225900: 76368

Sequence Region (SNP highlighted); tggtagccatgaagaaaaacaccaatc[g/a]ggagcctcagtggata (SEQ ID NO:14)

The allele frequencies;

| | Allele G | Allele A |
|---|---|---|
| Graves' disease | 42.7% (322/754) | 57.3% (432/754) |
| Control | 35.0% (463/1324) | 65.0% (861/1324) |

odds ratio = 1.39, χ² = 12.2, p = 4.7 × 10⁻⁴

The genotype frequencies;

| | GG | GA | AA |
|---|---|---|---|
| Graves' disease | 16.7% (63/377) | 52.0% (196/377) | 31.3% (118/377) |
| Control | 11.5% (76/662) | 47.0% (311/662) | 41.5% (275/662) |

genotype risk ratio = 1.55, χ² = 12.8, p = 1.7 × 10⁻³

17) 7134G>A (JO37-2)

Position (bp) in contig AF225900: 76410

Sequence Region (SNP highlighted); gatagtatatcatttccactcctctaaac[g/a]tctttagagagattactctttttcata gtt (SEQ ID NO:15)

The allele frequencies;

| | Allele G | Allele A |
|---|---|---|
| Graves' disease | 43.3% (323/746) | 56.7% (423/746) |
| Control | 34.8% (445/1278) | 65.2% (833/1278) |

odds ratio = 1.43, χ² = 14.4, p = 1.5 × 10⁻⁴

The genotype frequencies;

| | GG | GA | AA |
|---|---|---|---|
| Graves' disease | 17.4% (65/373) | 51.7% (193/373) | 30.8% (115/373) |
| Control | 11.9% (76/639) | 45.9% (293/639) | 42.3% (270/639) |

genotype risk ratio = 1.56, χ² = 15.0, p = 5.7 × 10⁻⁴

18) 7982A>G (JO36)

Position (bp) in contig AF225900: 77258

Sequence Region (SNP highlighted); cccaaattttgcctccaccgtcagatttgctgacactttaagctc[a/g]tggatttctcc tcttttgtttcatagctatac(SEQ ID NO:16)

The allele frequencies;

| | Allele G | Allele A |
|---|---|---|
| Graves' disease | 62.6% (468/748) | 37.4% (280/748) |
| Control | 70.4% (924/1312) | 29.6% (388/1312) |

odds ratio = 1.42, χ² = 13.4, p = 2.5 × 10⁻⁴

The genotype frequencies;

| | GG | GA | AA |
|---|---|---|---|
| Graves' disease | 37.2% (139/374) | 50.8% (190/374) | 12.0% (45/374) |
| Control | 49.4% (324/656) | 42.1% (276/656) | 8.5% (56/656) |

genotype risk ratio = 1.47, χ² = 14.9, p = 5.8 × 10⁻⁴

19) 8173T>C (JO35)

Position (bp) in contig AF225900: 77449

Sequence Region (SNP highlighted); ggggaactaggacatccaggaccgtttt[t/c]catacagaacccatctgtgttttcttag gcagtcccagctt(SEQ ID NO:17)

The allele frequencies;

| | Allele T | Allele C |
|---|---|---|
| Graves' disease | 37.0% (281/760) | 63.0% (479/760) |
| Control | 29.1% (389/1336) | 70.9% (947/1336 |

odds ratio = 1.43, χ² = 13.7, p = 2.1 × 10⁻⁴

The genotype frequencies;

| | TT | TC | CC |
|---|---|---|---|
| Graves' disease | 12.1% (46/380) | 49.7% (189/380) | 38.2% (145/380) |
| Control | 8.1% (54/668) | 42.1% (281/668) | 49.9% (333/668) |

genotype risk ratio = 1.57, χ² = 14.5, p = 6.9 × 10⁻⁴

20) 8857A>G (JO34)

Position (bp) in contig AF225900: 78133

Sequence Region (SNP highlighted); ttccttggctacatgctgggataggggctcat[a/g]gtaagtttgccagattcaaccaaa aaacgccacaaaa(SEQ ID NO:18)

The allele frequencies;

| | Allele G | Allele A |
|---|---|---|
| Graves' disease | 62.4% (469/752) | 37.6% (283/752) |
| Control | 71.0% (930/1310) | 29.0% (380/1310) |

odds ratio = 1.60, χ² = 24.4, p = 9.7 × 10⁻⁷

The genotype frequencies;

| | GG | GA | AA |
|---|---|---|---|
| Graves' disease | 37.2% (140/376) | 50.3% (189/376) | 12.5% (47/376) |
| Control | 49.9% (327/655) | 42.1% (276/655) | 7.9% (52/655) |

genotype risk ratio = 1.66, χ² = 17.2, p = 1.9 × 10⁻⁴

21) 10242G>T (JO31)

Position (bp) in contig AF225900: 79518

Sequence Region (SNP highlighted); gcaaaacgctgccaataaacagtctgtcagcaaagcc[g/t]gcagtacactgagaaagct cctattgccactg(SEQ ID NO:19)

The allele frequencies;

| | Allele T | Allele G |
|---|---|---|
| Graves’ disease | 38.4% (289/752) | 61.6% (463/752) |
| Control | 49.8% (632/1268) | 50.2% (636/1268) |

odds ratio = 1.59, χ² = 24.4, p = 6.4 × 10⁻⁷

The genotype frequencies;

| | TT | TG | GG |
|---|---|---|---|
| Graves’ disease | 14.4% (54/376) | 48.1% (181/376) | 37.5% (141/376) |
| Control | 26.5% (168/634) | 46.7% (296/634) | 26.8% (170/634) |

genotype risk ratio = 1.64, χ² = 24.6, p = 4.4 × 10⁻⁶

22) 10717G>A (JO30)

Position (bp) in contig AF225900: 79993

Sequence Region (SNP highlighted); ttgggaggcccaggcgggcggacctcttgaggtcaggagttc[g/a]agaccagcctggcc aacatggtgaaac(SEQ ID NO:20)

The allele frequencies;

| | Allele G | Allele A |
|---|---|---|
| Graves’ disease | 59.4% (442/744) | 40.6% (302/744) |
| Control | 50.0% (656/1312) | 50.0% (656/1312) |

odds ratio = 1.46, χ² = 16.9, p = 4.0 × 10⁻⁵

The genotype frequencies;

| | GG | GA | AA |
|---|---|---|---|
| Graves’ disease | 35.2% (131/372) | 48.4% (180/372) | 16.4% (61/372) |
| Control | 25.9% (170/656) | 48.2% (316/656) | 25.9% (170/656) |

genotype risk ratio = 1.55, χ² = 16.6, p = 2.5 × 10⁻⁴

23) 12311T>C (JO27-1)

Position (bp) in contig AF225900: 81587

Sequence Region (SNP highlighted); ggtagctatgcataagtaatttctaccagaagttgaagtgtaggaa[t/c]atctggggtc aaagcaaaaaaagactttccctg(SEQ ID NO:21)

The allele frequencies;

| | Allele T | Allele C |
|---|---|---|
| Graves’ disease | 58.6% (441/752) | 41.4% (311/752) |
| Control | 48.4% (626/1294) | 51.6% (668/1294) |

odds ratio = 1.51, χ² = 20.1, p = 7.4 × 10⁻⁶

The genotype frequencies;

| | TT | TC | CC |
|---|---|---|---|
| Graves’ disease | 34.0% (128/376) | 49.2% (185/376) | 16.8% (63/376) |
| Control | 24.3% (157/647) | 48.2% (312/647) | 27.5% (178/647) |

genotype risk ratio = 1.61, χ² = 19.9, p = 4.8 × 10⁻⁵

24) 16558T>C (JO18)

Position (bp) in contig AF225900: 85834

Sequence Region (SNP highlighted); atttcttttaccttttctttattttcccgtcag[t/c]aaatatttgttaggcaaataaga gcc (SEQ ID NO:22)

The allele frequencies;

| | Allele T | Allele C |
|---|---|---|
| Graves’ disease | 57.2% (396/692) | 42.8% (296/692) |
| Control | 48.7% (634/1302) | 51.3% (668/1302) |

odds ratio = 1.41, χ² = 13.2, p = 2.8 × 10⁻⁴

The genotype frequencies;

| | TT | TC | CC |
|---|---|---|---|
| Graves’ disease | 33.2% (115/346) | 48.0% (166/346) | 18.8% (65/346) |
| Control | 24.0% (156/651) | 49.5% (322/651) | 26.6% (173/651) |

genotype risk ratio = 1.58, χ² = 13.0, p = 1.5 × 10⁻³

25) 19178T>C (JO13)

Position (bp) in contig AF225900: 88454

Sequence Region (SNP highlighted); cattactcagatttcctatctccttggcaaatctggtcaccacaa[t/c]actctttaaaa aacacgctcatgttattagtatgaa (SEQ ID NO:23)

The allele frequencies;

| | Allele T | Allele C |
|---|---|---|
| Graves’ disease | 64.6% (406/628) | 35.4% (222/628) |
| Control | 71.9% (897/1248) | 28.1% (351/1248) |

odds ratio = 1.40, χ² = 10.3, p = 1.3 × 10⁻³

The genotype frequencies;

| | TT | TC | CC |
|---|---|---|---|
| Graves’ disease | 33.2% (115/346) | 48.0% (166/346) | 18.8% (65/346) |
| Control | 24.0% (156/651) | 49.5% (322/651) | 26.6% (173/651) |

genotype risk ratio = 1.63, χ² = 4.4, p = 3.6 × 10⁻²

26) 21660T>C (J08-2)

Position (bp) in contig AF225900: 90936

Sequence Region (SNP highlighted); agaaagacccaaacccacttttataccaaacccac[t/c]cttgtgataacaaa (SEQ ID NO:24)

The allele frequencies;

| | Allele T | Allele C |
|---|---|---|
| Graves’ disease | 57.3% (400/698) | 42.7% (298/698) |
| Control | 48.8% (616/1262) | 51.2% (646/1262) |

odds ratio = 1.4, χ² = 13.0, p = 3.1 × 10⁻⁴

The genotype frequencies;

| | TT | TC | CC |
|---|---|---|---|
| Graves’ disease | 32.7% (114/349) | 49.3% (172/349) | 18.1% (63/349) |
| Control | 23.5% (148/631) | 50.7% (320/631) | 25.8% (163/631) |

genotype risk ratio = 1.58, χ² = 13.1, p = 1.4 × 10⁻³

27) 22616A>G (JO6-1)

Position (bp) in contig AF225900: 91892

Sequence Region (SNP highlighted); tatctc[a/g]ctttcacacaagaagagatgtagaagattttatttgaagtgtgtctta (SEQ ID NO:25)

The allele frequencies;

| | Allele A | Allele G |
|---|---|---|
| Graves’ disease | 65.3% (443/678) | 34.7% (235/678) |
| Control | 72.9% (868/1190) | 27.1% (322/1190) |

odds ratio = 1.43, χ² = 11.9, p = 5.5 × 10⁻⁴

The genotype frequencies;

| | AA | AG | GG |
|---|---|---|---|
| Graves’ disease | 41.3% (140/339) | 48.1% (163/339) | 10.6% (36/339) |
| Control | 52.8% (314/595) | 40.3% (240/595) | 6.9% (41/595) |

genotype risk ratio = 1.61, χ² = 12.5, p = 1.9 × 10⁻³

28) 24212C>A (J03)

Position (bp) in contig AF225900: 93488

Sequence Region (SNP highlighted); ttcaaataactatttcaaaatattaaccctcaacaac[c/a]gtaatggatataaacgcat gtgctcatcaaaatggcagcaag(SEQ ID NO:26)

The allele frequencies;

| | Allele C | Allele A |
|---|---|---|
| Graves’ disease | 56.8% (407/716) | 43.2% (309/716) |
| Control | 48.2% (627/1302) | 51.8% (675/1302) |

odds ratio = 1.42, χ² = 14.0, p = 1.9 × 10⁻⁴

The genotype frequencies;

| | CC | CA | AA |
|---|---|---|---|
| Graves’ disease | 33.0% (118/358) | 47.8% (171/358) | 19.3% (69/358) |
| Control | 23.5% (153/651) | 49.3% (321/651) | 27.2% (177/651) |

genotype risk ratio = 1.60, χ² = 13.7, p = 1.0 × 10⁻³

T1DM Family Typing Analysis

384 UK affected sibling pair families were obtained from the British Diabetic Association Warren Repository (Davies (1994) Nature 371:130-136). 287 US affected sibling pair families were obtained from the Human Biological Database Interchange (Lernmark et al (1990) Am J.Hum. Gen. 47:1028-1030) and each had at least one affected sibling that had been diagnosed at age less than 29 years.

Five SNPs have been typed in UK families showing inherited T1DM; -23327G>C (MH30), -11534T>C (MH18), -1765T>C (CT50), 49G>A (CT42), and 6230G>A (CT60).

Nine SNPs have been typed in USA families showing inherited T1DM; -23327G>C(MH30), -11534T>C (MH18), -2871G>A (MH2), 2201C>T (MH1), -1765T>C (CT50), -1577 G>A (CT53), 49G>A (CT42), 6230G>A (CT60), and 10242G>T (JO31).

Allelic Association, transmission, from heterozygous parents to both affected and unaffected offspring, of single SNP alleles and of several marker haplotypes was assessed by basing on TDT (Spielman et al (1993) Am.J.Hum.Genet 52:487-498). The transmission disequilibrium test (TDT) examines the transmission of alleles or haplotypes from heterozygous parents and assesses preferential transmission of one allele or haplotype over the other. The test assumes an allele or haplotype associated with disease should be transmitted to affected children more often than would be expected by chance.

The data in families were analysed and the haplotype frequencies were calculated using TDTphase (ftp://ftp-gene.cimr.cam.ac.uk/pub/software; Dudbridge et al). The data in case-control study were analysed and the haplotype frequencies were calculated using casecon (ftp://ftp-gene.cimr.cam.ac.uk/pub/software; Dudbridge et al) which estimates haplotype frequencies using the EM algorithm. This software well known in the art and is available on the internet (ftp://ftp-gene.cimr.cam.ac.uk/pub/software; Dudbridge et al).

Confirmatory Association Analysis

To confirm the results of the preliminary study, a secondary analysis was performed. Key markers were retyped as described below in this secondary analysis to confirm the original typing results and resolve potential uncertainties.

-23327G>C (MH30) and 6230G>A (CT60) in UK T1DDM, and MH30 in Graves' cases and controls were re-typed using Invader™ SNP biplex assays (Third Wave Technologies Inc, Madison, WI). 34563C>T (AF343). was also typed using a Invader™ SNP biplex assay.

6230G>A (CT60) and 10242G>T (JO31) were retyped in Graves′ cases and controls using TaqMan™ probes (Perkin Elmer Applied Biosystem, Foster City, Calif.),

-1577 G>A (CT53) was retyped using the Invader™ SNP uniplex assay used in the original analysis.

Controls which failed to yield the original typing results on retyping were excluded from the revised data shown in FIG. 1.

Results

Three SNPs have been typed in both UK and USA families showing inherited T1DM; -23327G>C (MH30), 49G>A (CT42), and 6230G>A (CT60). -23327G>C (MH30) was found to show disease association in both UK and USA families, and 6230G>A (CT60) was found to show disease association in UK families.

The known associated SNP in this locus at 49 (CT42) (Kristiansen et al (2000) Genes and Immunity 1: 170-184) does not show the disease association in both families with TLDM in this study.

FIG. 2 shows the association.of each marker and of a haplotype of two markers in this locus at -23327 (MH30) and 6230 (CT60) in both UK and USA families with T1DM. This data confirms the results of the preliminary data analysis shown in FIG. 4.

The measure of association in FIG. 2 is the percentage transmission of an allele of a haplotype from parent to child (which is 50% in non-disease associated alleles or a haplotype) (a), together with -log(p value) (b). Markers and a haplotype of two markers in this locus at 23327 (MH30) and 6230 (CT60) show strong disease association in FIG. 2.

Preliminary results for the association of the haplotype in UK families with T1DM is shown in Table 3a. A haplotype (1, 1) of two markers in this locus at -23327 (MH30) and 6230 (CT60), which comprises nucleotide G at these positions, is strongly associated with T1DM. The results of a confirmatory analysis which also included US families with T1DM are shown in Table 3b. In this analysis, key markers were also retyped to resolve uncertainties and confirm the original typing results. The secondary analysis confirms the association observed in the original analysis.

Other haplotypes of the two markers in this locus at -23327 (MH30) and 6230 (CT60) are protective haplotypes in UK families with T1DM.

Preliminary analysis of the association of the haplotype in UK cases with T1DM and Graves' disease is shown in Table 4a. A confirmatory analysis, in which key markers were retyped to confirm the original typing results and revise uncertainties, is shown in Table 4b. This secondary analysis of the data confirms the association of the haplotypes observed in the original analysis.

These results show that a haplotype (1, 1) of two markers in this locus at -23327 (MH30) and 6230 (CT60), is strongly associated with both TlDM and Graves' disease. Frequencies of haplotypes are similar in both diseases, indicating that this haplotype is the common disease susceptibility haplotype for both diseases.

The marker at -34563 (AF343) showed strong linkage disequilibrium with the marker at -23327 (MH30) in both cases and controls (Table 5a and Table 5b), as indicated by a high D′value (when the value D′=0 means no LD, D′=1 means complete LD).

The association of haplotypes of markers -34563(AF343), 23327 (MH30) and 6230 (CT60) with Grave's disease is shown in Table 6. The (2, 1, 1) haplotype of these markers was shown to be strongly associated with Graves' disease.

The SNPs described herein are therefore strongly associated with Grave's Disease. In certain populations type I diabetes has not been previously associated with CTLA-4. However, from the data presented herein, this association is as strong as that in Graves disease.

Therefore, the SNPs and haplotypes described herein are effective risk markers for T-cell associated autoimmune disorders such as Grave's disease and Type 1 diabetes.

TABLE 1

| SEQ ID | Locus name | Assay name | Allele 1 | Allele 2 | Position on AF225900 |
|---|---|---|---|---|---|
| 1 | −34563T > C | AF343 | C | T | 34714 |
| 2 | −23327G > C | MH30 | G | C | 45950 |
| 3 | −14953G > T | MH26 | T | G | 54324 |
| 4 | −12527G > A | MH20 | G | A | 56750 |
| 5 | −11534T > C | MH18 | T | C | 57743 |
| 6 | −10914T > C | MH17 | T | C | 58363 |
| 7 | −9914G > T | MH15 | T | G | 59363 |
| 8 | −8916G > A | MH13-1 | G | A | 60361 |
| 9 | −2871G > A | MH2 | G | A | 66406 |
| 10 | −2201C > T | MH1 | T | C | 67076 |
| 11 | −1765T > C | CT50 | T | C | 67512 |
| 12 | −1577G > A | CT53 | G | A | 67700 |
| 13 | 6230G > A | CT60 | G | A | 75506 |
| 14 | 7092G > A | JO37-3 | G | A | 76368 |
| 15 | 7134G > A | JO37-2 | G | A | 76410 |
| 16 | 7982A > G | JO36 | G | A | 77258 |
| 17 | 8173T > C | JO35 | T | C | 77449 |
| 18 | 8857A > G | JO34 | G | A | 78133 |
| 19 | 10242G > T | JO31 | T | G | 79518 |
| 20 | 10717G > A | JO30 | G | A | 79993 |
| 21 | 12311T > C | JO27-1 | T | C | 81587 |
| 22 | 16558T > C | JO18 | T | C | 85834 |
| 23 | 19178T > C | JO13 | T | C | 88454 |
| 24 | 21660T > C | JO8-2 | T | C | 90936 |
| 25 | 22616A > G | JO6-1 | G | A | 91892 |
| 26 | 24212C > A | JO3 | C | A | 93488 |

TABLE 2

| Previous known as | names in this report |
|---|---|
| C to T transition at position −318 | −319C > T |
| of the promoter sequence¹⁾ | |
| G to A transition at position 49 of | 49G > A (CT42) |
| exon 1²⁾ | |
| T to C transition in intron 1 at 819 bp | 1822T > C |
| upstream of the exon 2 start site³⁾ | |

¹⁾Deichmann et al (1996) Biochem. Biophys. Res. Commun. 225: 817-818
²⁾Harper et al (1991) J. Immunol 147: 1037-44
³⁾Marron et al (2000) Diabetes 49: 492-499

TABLE 3a

| Haplotypes | T | NT | % T | P |
|---|---|---|---|---|
| (1, 1) | 123 | 59 | 67.6 | 0.000035 |
| (2, 2) | 59 | 96 | 38.1 | 0.0084 |
| (1, 2) | 18 | 40 | 31.0 | 0.0068 |
| (2, 1) | 8 | 13 | 38.1 | 0.297 |

TABLE 3b

| Haplotypes | T | NT | % T | p |
|---|---|---|---|---|
| (1, 1) | 462 | 353 | 56.7 | 0.000931 |
| (2, 2) | 349 | 441 | 44.2 | 0.00433 |
| (1, 2) | 32 | 39 | 45.1 | 0.409 |
| (2, 1) | 8 | 18 | 30.8 | 0.0499 |

TABLE 4a

| | T1DM | | Graves′ | | healthy | |
|---|---|---|---|---|---|---|
| | cases | | cases | | controls | |
| haplotypes | n | % | N | % | n | % |
| (1, 1) | 304 | 60.1 | 456 | 62.1 | 623 | 50.6 |
| (2, 2) | 160 | 31.7 | 251 | 34.2 | 559 | 45.4 |
| (1, 2) | 24 | 4.7 | 17 | 2.3 | 42 | 3.4 |
| (2, 1) | 18 | 3.5 | 10 | 1.4 | 6 | 0.5 |

p (T1DM vs control): 1.5 × 10⁻⁹
p (Graves′ vs control): 7.3 × 10⁻⁷
p (T1DM vs Graves′): 0.01

TABLE 4b

| | T1DM | | Graves′ | | healthy | |
|---|---|---|---|---|---|---|
| | cases | | cases | | controls | |
| Haplotypes | n | % | N | % | n | % |
| (1, 1) | 437 | 63.1 | 481 | 62.6 | 675 | 52.0 |
| (2, 2) | 236 | 34.1 | 266 | 34.6 | 578 | 44.5 |
| (1, 2) | 16 | 2.3 | 15 | 2.0 | 39 | 3.0 |
| (2, 1) | 3 | 0.4 | 6 | 0.8 | 6 | 0.5 |

p (T1DM vs control): 4.0 × 10−5
p (Graves′ vs control): 2.1 × 10−5
p (T1DM vs Graves′): 0.8

TABLE 5a

| | |
|---|---|
| Graves' | Healthy |
| cases | controls |

| haplotypes | N | % | N | % |
|---|---|---|---|---|
| (1, 1) | 1.2 | 0.2 | 1.2 | 0.1 |
| (2, 2) | 129.2 | 17.2 | 238.2 | 18.7 |
| (1, 2) | 137.8 | 18.3 | 336.8 | 26.5 |
| (2, 1) | 483.8 | 64.3 | 695.8 | 54.7 |

p = 6.3 × 10⁻⁵

TABLE 5b

| | Control | Case |
|---|---|---|
| D′ | 0.993 | 0.986 |

TABLE 6

| | |
|---|---|
| Graves' cases | Healthy controls |

| Haplotypes | N | % | n | % |
|---|---|---|---|---|
| (2, 1, 1) | 472.8 | 62.9 | 657.6 | 51.7 |
| (2, 2, 2) | 126.1 | 16.8 | 234.3 | 18.4 |
| (1, 2, 2) | 134.8 | 17.9 | 334.5 | 26.3 |
| (others) | 18.2 | 2.4 | 45.6 | 3.6 |

P = 3.6 × 10⁻⁶

1. A method for determining the susceptibility of an individual to a T-cell associated autoimmune disorder comprising:
determining the identity of a nucleotide present at one or more positions of single nucleotide polymorphism within the CTLA-4 locus of a genomic DNA sequence obtained from an individual, said one or more positions being selected from the group consisting of;
positions, -34563, -23327, -14953, -12527, -11534, -10914, -9914, -8916, -2871, -2201, -1765, -1577, 6230, 7092, 7134, 7982, 8173, 8857, 10242, 10717, 12311, 16558, 19178, 21660, 22616 and 24212 of the CTLA-4 locus, wherein the nucleotide 5′ to the A of the ATG of the CTLA-4 initiation codon is designated -1 and the sequence of the CTLA-4 locus has the database accession number AF225900.

2. A method according to claim 1 wherein the susceptibility of the individual to a T-cell mediated autoimmune disorder is indicated by the identity of the nucleotide present at said one or more positions.

3. A method according to claim 2 wherein the T cell mediated autoimmune disorder is Graves disease or Type 1 Diabetes Mellitus.

4. A method according to any one of claims 1 to 3 wherein the identity of the nucleotide at the one or more positions of single nucleotide polymorphism is shown in Table 1 and SEQ ID NOS: 1 to 26.

5. A method according to any one of the preceding claims comprising determining the identity of the nucleotides present at positions -23327 and 6230 within the CTLA-4 locus.

6. A method according to claim 5 comprising additionally determining the identity of the nucleotide present at position -34563 within the CTLA-4 locus.

7. A method according to claim 5 comprising determining the presence of a haplotype which comprises nucleotide G at positions -23327 and 6230 of the CTLA-4 locus.

8. A method according to any one of the preceding claims comprising determining the binding of a oligonucleotide probe to a genomic DNA sample, the probe comprising a nucleotide sequence which binds specifically to a particular allele of the one or more single nucleotide polymorphisms and does not bind specifically to other alleles of the one or more single nucleotide polymorphisms.

9. A method according to claim 7 wherein the oligonucleotide probe comprises a label and binding of the probe is determined by detecting the presence of the label.

10. A method according to any one of the preceding claims comprising amplifying a region of a genomic DNA sample containing the one or more positions of single nucleotide polymorphisms.

11. A method according to claim 9 wherein the region of genomic sample is amplified using a pair of oligonucleotide primers; the first member of the pair comprising a nucleotide sequence which hybridises to a complementary sequence which is proximal to and 5′ of the at least one polymorphism, the second member of the primer pair comprising a nucleotide sequence which hybridises to a complementary sequence which is proximal to and 3′ of the at least one polymorphism.

12. A method according to claim 10 wherein the region of genomic sample is amplified using a pair of oligonucleotide primers, the first member of the pair comprising a nucleotide sequence which hybridises to a complementary sequence which is proximal to and 5′ or 3′ of the at least one polymorphism, the second member of the pair comprising a nucleotide sequence which hybridises under stringent conditions to a particular allele of the at least one polymorphism and not to other alleles such that amplification only occurs in the presence of the particular allele.

13. A method according to claim 10 or claim 11 wherein the amplified region of genomic sample is sequenced and the allele of the at least one polymorphism in the genomic sample is determined.

14. A method according to claim 11 or claim 12 comprising determining the binding of a oligonucleotide probe to the amplified region of the genomic sample, the probe comprising a nucleotide sequence which binds specifically to a particular allele of the one or more polymorphisms and does not bind specifically to other alleles of the one or more polymorphisms.

15. A method for determining the presence or absence of an allele of a polymorphic nucleic acid sequence in a test sample comprising:
contacting a polymorphic nucleic acid sequence with a probe which specifically binds to the allele of the polymorphic nucleic acid sequence; and, determining binding of the nucleic acid sequence and the probe,
said method being characterised in that the polymorphic nucleic acid sequence comprises one or more positions of single nucleotide polymorphism selected from the group consisting of positions -34563, -23327, -14953, -12527, -11534, -10914, -9914, -8916, -2871, -2201, -1765, -1577, 6230, 7092, 7134, 7982, 8173, 8857, 10242, 10717, 12311, 16558, 19178, 21660, 22616 and 24212 of the CTLA-4 locus, wherein the nucleotide 5′ to the A of the ATG of the CTLA-4 initiation codon is designated -1 and the sequence of the CTLA-4 locus has the database accession number AF225900,
the identity of the nucleotide at the one or more positions of single nucleotide polymorphism determining the allele of the polymorphic nucleic acid sequence.

16. A method for determining the presence or absence in a test sequence of an allele of a polymorphic nucleic acid sequence comprising one or more positions of single nucleotide polymorphism, the method comprising:
determining the identity of the nucleotide at one or more positions of single nucleotide polymorphism selected from the group consisting of positions -34563, -23327, -14953, -12527, -11534, -10914, -9914, -8916, -2871, -2201, -1765, -1577, 6230, 7092, 7134, 7982, 8173, 8857, 10242, 10717, 12311, 16558, 19178, 21660, 22616 and 24212 of the CTLA-4 locus wherein the nucleotide 5′ to the A of the ATG of the CTLA-4 initiation codon is designated -1 and the sequence of the CTLA-4 locus has the database accession number AF225900, the presence of the allele of the polymorphic nucleic acid sequence being determined by the identity of the nucleotide at the one or more positions of single nucleotide polymorphism.

17. A method according to claim 16 comprising determining the identity of the nucleotide at positions -23327 and 6230 of the CTLA-4 locus.

18. A method according to claim 17 additionally comprising determining the identity of the nucleotide at position -34563 of the CTLA-4 locus.

19. A method according-to claim 17 comprising determining the presence or absence of nucleotide G at positions -23327 and 6230 of the CTLA-4 locus.

20. An oligonucleotide which hybridises specifically to an allele of a nucleic acid sequence which comprises one or more positions of single nucleotide polymorphism selected from the group consisting of positions -34563, -23327, -14953, -12527, -11534, -10914, -9914, -8916, -2871, -2201, -1765, -1577, 6230, 7092, 7134, 7982, 8173, 8857, 10242, 10717, 12311, 16558, 19178, 21660, 22616, 24212 of CTLA-4.

21. An oligonucleotide according to claim 20 comprising a label.

22. A pair of amplification primers having a first member comprising a nucleotide sequence which hybridises to a complementary sequence which is proximal to and 5′ of the one or more positions of single nucleotide polymorphism selected from the group consisting of positions -34563, -23327, -14953, -12527, -11534, -10914, -9914, -8916, -2871, -2201, -1765, -1577, 6230, 7092, 7134, 7982, 8173, 8857, 10242, 10717, 12311, 16558, 19178, 21660, 22616, 24212 of CTLA-4, the second member of the primer pair comprising a nucleotide sequence which hybridises to a complementary sequence which is proximal to and 3′ of said one or more positions of single nucleotide polymorphism.

23. A pair of amplification primers having a first member comprising a nucleotide sequence which hybridises to a complementary sequence which is proximal to and 5′ or 3′ of one or more positions of single nucleotide polymorphism selected from the group consisting of positions -34563, -23327, -14953, -12527, -11534, -10914, -9914, -8916, -2871, -2201, -1765, -1577, 6230, 7092, 7134, 7982, 8173, 8857, 10242, 10717, 12311, 16558, 19178, 21660, 22616, 24212 of CTLA-4,
the second member of the pair comprising a nucleotide sequence which hybridises under stringent conditions to a particular allele of the one or more polymorphisms and not to other alleles such that amplification only occurs in the presence of the particular allele.

24. An assay kit comprising a pair of amplification primers according to claim 22 or claim 23 and/or an oligonucleotide according to claim 20 or claim 21.
